(19) United States
(12) Patent Application Publication
(10) Pub. No.: US 2024/0245885 A1
(43) Pub. Date: Jul. 25, 2024

(54) METHOD OF MANUFACTURING AN ELONGATED CATHETER HAVING MULTIPLE SENSORS FOR THREE-DIMENSIONAL LOCATION OF THE CATHETER

(71) Applicant: Covidien LP, Mansfield, MA (US)

(72) Inventors: Nathan J. Knutson, Long Lake, MN (US); Oren P. Weingarten, Hod-Hasharon (IL)

(21) Appl. No.: 18/626,939

(22) Filed: Apr. 4, 2024

Related U.S. Application Data
(63) Continuation of application No. 16/824,172, filed on Mar. 19, 2020, now Pat. No. 11,975,157.
(60) Provisional application No. 62/833,166, filed on Apr. 12, 2019.

(51) Int. Cl. A61M 25/00; A61B 34/35; A61M 25/01

(52) U.S. Cl. CPC A61M 25/005; A61M 25/0012; A61M 25/0105; A61M 25/0136; A61B 34/35; A61M 2025/0166; A61M 2025/0175

(57) ABSTRACT

A flexible catheter includes an elongated body, a first monolithic wire, and a second monolithic wire. The first monolithic wire forms a first sensor that is disposed in the distal end portion of the elongated body. The first sensor is configured to detect the position of a distal end of the elongated body within anatomy of a patient. The second monolithic wire forms a second sensor that is disposed in the distal end portion of the elongated body a known distance from the first sensor. The first and second sensors are configured to determine the position of the distal end portion within six degrees-of-freedom. A method of manufacturing the flexible catheter is also provided.

### CROSS-REFERENCE TO RELATED APPLICATION

This application is a continuation of U.S. patent application Ser. No. 16/824,172 filed Mar. 19, 2020, now allowed, which claims the benefit of and priority to U.S. Provisional Application No. 62/833,166, filed on Apr. 12, 2019, the entire contents of which are incorporated by reference herein.

### BACKGROUND

### 1. Technical Field

The disclosure relates to elongated catheters and, more specifically, to methods of manufacturing elongated catheters including sensors for three-dimensional location of the catheter.

### 2. Discussion of Related Art

A common interventional procedure in the field of pulmonary medicine is bronchoscopy, in which a bronchoscope is inserted into the airways through the patient's nose or mouth. The structure of a bronchoscope generally includes a long, thin, flexible tube that typically contains three elements: an illumination assembly for illuminating the region distal to the bronchoscope's tip via an optical fiber connected to an external light source; an imaging assembly for delivering back a video image from the bronchoscope's distal tip; and a lumen or working channel through which instruments may be inserted, including but not limited to placement (e.g., guide wires), diagnostic (e.g., biopsy tools) and therapeutic (e.g., treatment catheters or laser, cryogenic, radio frequency, or microwave tissue treatment probes) instruments.

During particular procedures (e.g., microwave ablation and biopsy) a catheter having an extended working channel may be inserted through a working channel of a bronchoscope to enable navigation to sites too remote and having luminal diameters too small for the bronchoscope. Generally, a locatable guide is positioned at a distal end of the extended working channel to guide the catheter to targeted tissue. When the distal end of the extended working channel is positioned adjacent targeted tissue, the locatable guide is removed from the extended working channel. Then, an instrument may be inserted through the extended working channel in order to act on the targeted tissue (e.g., perform a biopsy or ablation of the targeted tissue). When the locatable guide is removed, the location of the catheter is no longer visible.

### SUMMARY

In an aspect of the disclosure, a flexible catheter includes an elongated body, a first monolithic wire, and a second monolithic wire. The elongated body has a proximal end portion, a distal end portion, and a wall that defines a working channel therethrough. The first monolithic wire forms a first sensor that is disposed in the distal end portion of the elongated body. The first sensor is configured to detect the position of a distal end of the elongated body within anatomy of a patient. The second monolithic wire forms a second sensor that is disposed in the distal end portion of the elongated body a known distance from the first sensor. The first and second sensors are configured to determine the position of the distal end portion within six degrees-of-freedom.

In aspects, the first sensor is formed from turns of the first wire about the working channel in the distal end portion. The first wire may include first and second leads that form a first twisted pair proximal of the turns. The first twisted pair may extend to a proximal end portion of the elongated body. The second sensor may be formed from a coil of the second wire. The second wire may include third and fourth leads that form a second twisted pair proximal of the coil. The second twisted pair may extend to a proximal end portion of the elongated body. The first twisted pair may be helically wound about the working channel between the first sensor and the proximal end portion of the elongated body. The coil may be disposed within the wall of the elongated body and positioned between two adjacent helical wraps of the first twisted pair. The second twisted pair may be helically wound about the working channel between the second sensor and the proximal end portion of the elongated body. The second twisted pair may be interleaved with the first twisted pair along the elongated body.

In some aspects, the first and second wires are disposed entirely within the wall of the elongated body distal of the proximal end portion. The elongated body may include a braid that defines the working channel and an outer coating disposed over the braid for isolating the working channel from an environment surrounding the flexible catheter. The first and second twisted pairs may follow the braid such that a thickness of the wall is smooth between the proximal and distal end portions of the elongated body.

In certain aspects, the flexible catheter is substantially rigid between the first and second sensors. The second sensor may be configured to determine the orientation of the distal end portion. Alternatively, the flexible catheter may flexible between the first and second sensors. The second sensor may be configured to determine the deflection of the distal end portion between the first and second sensors. The second sensor may be configured to determine an arc of the catheter between the second sensor and the first sensor. The flexible catheter may further include a third monolithic wire forming a third sensor disposed on the elongated body a known distance proximally of the second sensor. The third sensor may be configured to determine an arc of the catheter between the third sensor and the second sensor.

In another aspect of the disclosure, a catheter system includes a flexible catheter, and a handle. The flexible catheter includes an elongated body, a first monolithic wire, and a second monolithic wire. The elongated body has a proximal end portion and a distal end portion. The elongated body has a wall that defines a working channel therethrough. The first monolithic wire forms a first sensor disposed in the distal end portion of the elongated body. The first sensor is configured to detect the position of a distal end of the elongated body within an anatomy of a patient. The second monolithic wire is forms a second sensor that is disposed in the distal end portion of the elongated body a known distance from the first sensor. The handle is disposed over the proximal end portion of the elongated body. The handle is longitudinally and rotatably fixed to the flexible catheter. The handle includes an adapter in electrical communication with the first and second sensors. The first and second sensors are configured to determine the position of the distal end portion within six degrees-of-freedom.

In aspects, the catheter system includes a telescopic channel disposed over a portion of the elongated body and is coupled to a distal end of the handle.

In another aspect of the disclosure, a method of manufacturing a flexible catheter that is locatable within six degrees-of-freedom includes placing a first sensor, placing a second sensor, helically winding a first twisted pair of leads, helically winding a second twisted pair of leads, and forming an outer coating. Placing the first sensor includes the first sensor being formed of a first monolithic wire about a distal end portion of a working channel. Placing the second sensor includes the second sensor being formed of a second monolithic wire along the working channel a known distance from the first senor. Helically winding the first pair of twisted pair of leads includes the first twisted pair of leads being part of the first monolithic wire extending proximally from the first sensor to a proximal end portion of the working channel. Helically winding the second twisted pair of leads includes the second twisted pair of leads being part of the second monolithic wire extending proximally from the second sensors to the proximal end portion of the working channel such that the second twisted pair of lead is interleaved with the first twisted pair of leads. Forming the outer coating over the working channel embeds the first and second wires within a wall of the flexible catheter to have a constant diameter distal of the proximal end portion of the working channel.

In aspects, placing the first sensor about the distal end portion of the working channel includes winding the first monolithic wire about the distal end portion of the working channel. Placing the second sensor along the working channel may include placing the second sensor between two adjacent windings of the first twisted pair.

In some aspects, the method includes forming the second sensor by forming a coil in the second wire. Placing the second sensor along the working channel may include selecting the known distance such that the flexible catheter is rigid between the first and second sensors. Alternatively, placing the second sensor along the working channel may include selecting the known distance such that the flexible catheter is flexible between the first and second sensors.

In certain aspects, placing the second sensor along the working channel is subsequent to helically winding the first twisted pair of leads. Alternatively, helically winding the first pair of leads occurs simultaneously with helically winding the second pair of leads.

Further, to the extent consistent, any of the aspects described herein may be used in conjunction with any or all of the other aspects described herein.

### BRIEF DESCRIPTION OF THE DRAWINGS

Various aspects of the disclosure are described hereinbelow with reference to the drawings, which are incorporated in and constitute a part of this specification, wherein:

FIG. 1 is a side view of a catheter assembly provided in accordance with the disclosure;

FIG. 2 is a cross-sectional view of the catheter assembly of FIG. 1 taken along the section line 2-2 of FIG. 1;

FIG. 3 is a cross-section view of the catheter assembly of FIG. 1 taken along the section line 3-3 of FIG. 1;

FIG. 4 is a cross-section view of the catheter assembly of FIG. 1 taken along the section line 4-4 of FIG. 1

FIG. 5 is a side view of an inner liner of the catheter assembly of FIG. 1 over a mandrel;

FIG. 6 is a side view of a braid over the inner liner of FIG. 5;

FIG. 7 is an enlarged view of the area of detail of FIG. 6;

FIG. 8 is an enlarged view of the area of detail of FIG. 6;

FIG. 9 is a side view of wires turned over the braid of FIG. 6 towards a proximal end of the braid;

FIG. 10 is a side view of a first outer tube slid over a proximal end of the braid of FIG. 9;

FIG. 11 is a side view of an outer sleeve positioned over the first outer tube of FIG. 10;

FIG. 12 is a side view of the wires wrapped over a portion of the first outer tube and the outer sleeve of FIG. 11;

FIG. 13 is a side view of a second outer tube slid over a distal end of the braid, a portion of the wires, a portion of the first outer tube, and a portion of the outer sleeve of FIG. 12;

FIG. 14 is a side view of an outer coating formed over the braid from the first and second outer tubes with the wires unwrapped from over the outer sleeve and the outer sleeve removed from over the first outer tube of FIG. 13;

FIG. 15 is a side view of the catheter of FIG. 1 being cut to length from the braid, wires, and outer coating of FIG. 14;

FIG. 16 is a side view of a proximal end portion of the catheter of FIG. 15 positioned in a body of a handle assembly with a half-shell of the body removed;

FIG. 17 is a side view of the catheter assembly of FIG. 1 with the half-shell of the body removed;

FIG. 18 is a schematic illustration of a robotic surgical system configured for use in accordance with the disclosure;

FIG. 19 is a side view of a distal end portion of a prior art catheter having a single sensor;

FIG. 20 is a side view of the distal end portion of the catheter assembly of FIG. 1 where a second sensor is positioned a first distance from a first sensor; and

FIG. 21 is a side view of the distal end portion of the catheter assembly of FIG. 1 where a second sensor is positioned a second distance from a first sensor.

### DETAILED DESCRIPTION

Some catheters have been developed with a single sensor for locating a distal end of the catheter within the anatomy of a patient. Such catheters may be limited to obtaining the location of the catheter within five degrees-of-freedom. There is a need for a catheter having sensors capable of determining the location of the catheter within six degrees-of-freedom.

Accordingly, the disclosure generally provides a catheter having an Extended Working Channel (EWC) and a first sensor positioned at a distal end of the EWC for locating the distal end of the EWC within the anatomy of a patient and a second sensor spaced apart from the first sensor a known distance to determine the position of the first sensor relative to the second sensor such that the position of the distal end of the EWC is determinable within six degrees-of-freedom (DOF). Such a catheter eliminates the need for a separate locatable guide to be inserted through the EWC to locate the distal end of the catheter within the anatomy of a patient. In addition, such a catheter eliminates the need to remove the locatable guide before insertion of an instrument through the EWC to treat targeted tissue. Further, such a catheter is locatable during an entire procedure such that the changes in the location of the distal end of the catheter are detectable and/or the catheter is repositionable during the procedure when an instrument positioned within the EWC. In addition, as detailed herein, such a catheter may be manufactured without increasing a wall thickness of the catheter or an overall diameter of the catheter.

Embodiments of the disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is closest to the clinician and the term “distal” refers to the portion of the device or component thereof that is farthest from the clinician.

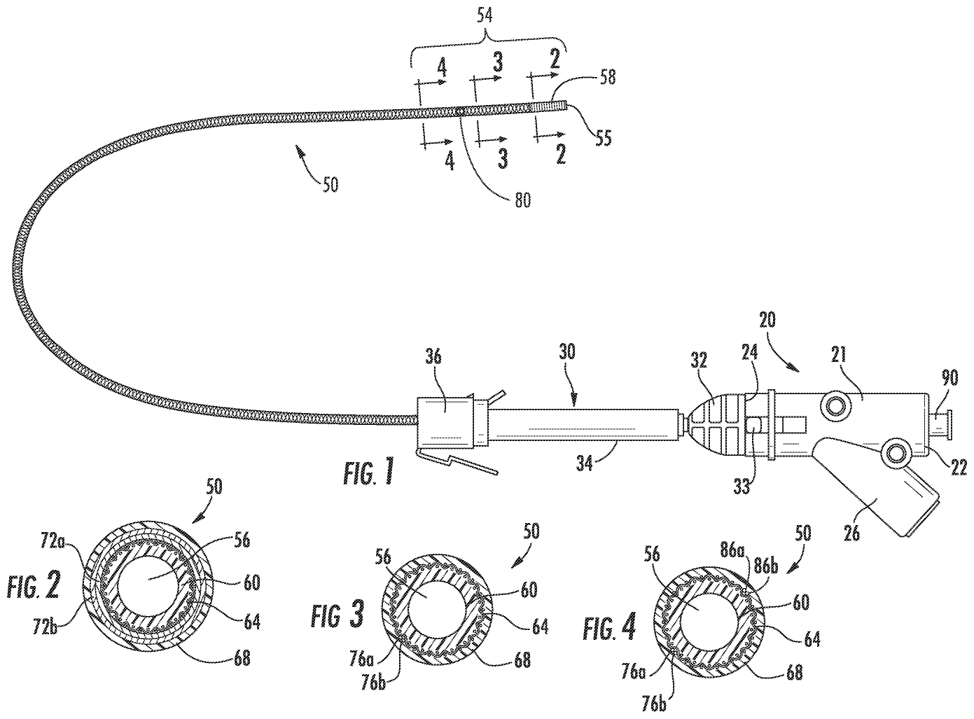

Referring now to FIG. 1, a catheter assembly 10 is provided in accordance with the disclosure and includes a handle assembly 20, a telescopic channel 30, and an elongated catheter 50 having a proximal portion 52 (FIG. 12) and a distal portion 54. The handle assembly 20 is positioned over the proximal portion 52 of the catheter 50 to permit a clinician to manipulate the catheter assembly 10. The telescopic channel 30 is positioned over the catheter 50 between the proximal portion 52 and the distal portion 54 to provide lateral support for the elongated catheter 50. The telescopic channel 30 includes a proximal or first end 32 that is coupled to a distal end 24 of the handle assembly 20 and a distal or second end 36 that is configured to couple the catheter assembly 10 to a bronchoscope (not shown). The telescopic channel 30 includes an extendable body portion 34 between the first and second ends 32, 36 that are expandable along a longitudinal axis and substantially rigid transverse to the longitudinal axis. The extendable body portion 34 allows the first end 32 to translate along and rotate about the longitudinal axis relative to the second end 36. When the first end 32 is coupled to the handle assembly 20, the proximal portion 52 of the catheter 50 translates and rotates with the first end 32 of the telescopic channel 30.

Examples of methods of marking the location of and registering a pathway to targeted tissue may be found in commonly assigned U.S. Patent Publication Nos. 2016/0000414 and 2016/0000356. Examples of guiding a catheter to and treating targeted tissue may be found in commonly assigned U.S. Pat. Nos. 9,459,770 and 9,247,992. Examples of bronchoscopes, handles, and support systems for microwave ablation catheters may be found in commonly assigned U.S. Pat. No. 9,867,665.

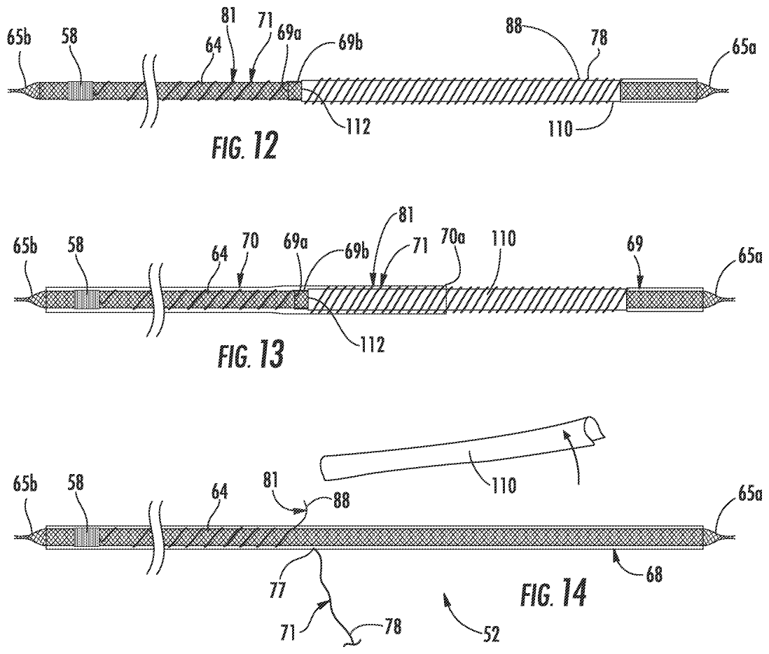

With additional reference to FIGS. 2 and 3, the catheter 50 has a proximal end 53 (FIG. 13) and a distal end 55 and defines an EWC 56 therebetween. The EWC 56 allows instruments (not shown) to be inserted through the catheter 50 to treat targeted tissue adjacent the distal end 55 of the catheter 50. The catheter 50 includes a first sensor 58 disposed at the distal portion 54 of the catheter 50 adjacent the distal end 55 of the catheter 50 such that the distal end 55 of the catheter 50 is locatable within the anatomy of a patient. The catheter 50 also includes a second sensor 80 disposed in the distal portion 54 of the catheter 50 disposed proximal of the first sensor 58.

With particular reference to FIGS. 2-4, the catheter 50 includes an inner liner 60, a braid 64, and an outer coating 68. The inner liner 60 defines the EWC 56 that passes entirely through the catheter 50. It is contemplated that the catheter 50 may be constructed without the inner liner 60 such that the braid 64 defines the EWC 56.

As described in greater detail below, the first sensor 58 is formed of a wire 71 (FIG. 6) turned over the braid 64 and covered by the outer coating 68 to form the first sensor 58. The wire 71 includes leads 76a, 76b that proximally follow the braid 64 to the proximal portion 52 (FIG. 12) of the catheter 50. It will be appreciated that while the portions of the wire 71 (e.g., the first sensor 58 and the leads 76a, 76b) are discussed individually herein, that the wire 71 is monolithically formed (i.e., the wire 71 is a continuous wire without any solder joints between different portions thereof). By forming the first sensor 58 and the leads 76a, 76b from a continuous monolithic wire, the useful life of the first sensor 58, and thus the catheter 50, may be increased.

The second sensor 80 is formed of a wire 81 that is coiled and covered by the outer coating 68. The wire 81 includes leads 86a, 86b that proximally follow the braid 64 to the proximal portion 52 of the catheter 50. It will be appreciated that while the portions of the wire 81 (e.g., the second sensor 80 and the leads 86a, 86b) are discussed individually herein, that the wire 81 is monolithically formed. By forming the second sensor 80 and the leads 86a, 86b from a continuous monolithic wire, the useful life of the second sensor 80, and thus the catheter 50, may be increased.

The inner liner 60 and the outer coating 68 are formed from polymer tubes, as detailed below, which are made from of a reflowable polymer material (e.g., Arnitel®, Hytrel®, Pebax®, polytetrafluoroethylene (PTFE)) which may bond to the braid 64, the wire 71, the wire 81, and to one another. The braid 64 is constructed of a mesh of between 16 and 32 of similar or varying material cords woven together (e.g., stainless steel, Dacron®, polyethylene naphthalate (PEN), polyethylene terephthalate (PET), and/or insulated electrical wire). The wires 71, 81 are solid core magnetic wire with a thin dielectric coating (e.g., a copper wire with a polyimide coating).

Referring now to FIGS. 5-15, the construction of the catheter 50 and a method of manufacturing the catheter assembly 10 will be described in accordance with the disclosure. Initially referring to FIG. 5, the inner liner 60 is slid over a mandrel 100. The mandrel 100 provides rigidity to the flexible components of the catheter 50 while the catheter 50 is assembled. The inner liner 60 has an inner diameter substantially equal to but slightly larger than an outer diameter of the mandrel 100 and has a length substantially equal to a length of the mandrel 100. The mandrel 100 may be coated with a PTFE coating to assist in sliding the inner liner 60 over the mandrel 100 and to prevent the inner liner 60 from bonding to the mandrel 100. The outer diameter of the mandrel 100 is substantially equal to a desired diameter of the EWC 56 (FIG. 2) and the length of the mandrel 100 is longer than a final desired length of the catheter 50. The mandrel 100 may have a diameter in a range of about 0.050 to about 0.100 inches (e.g., about 0.090 inches) and have a length in a range of about 30 to about 90 inches (e.g., about 62 inches).

With particular reference to FIG. 5, the braid 64 is formed over the inner liner 60 with portions of the braid 64 extending beyond the ends of the mandrel 100 such that the mandrel 100 and the inner liner 60 are completely within the braid 64. The ends 65a, 65b of the braid 64 that extend beyond the mandrel 100 may have a diameter less than the diameter of the mandrel 100. By having a diameter less than the mandrel 100, the ends 65a, 65b of the braid 64 may retain the mandrel 100 within the braid 64 during construction of the catheter 50. It is contemplated that the braid 64 may be performed and slid over the inner liner 60 and the mandrel 100.

The braid 64 is formed by helically weaving cords 66 of material over a cylinder (e.g., the inner liner 60 and the mandrel 100. The cords 66 define channels 67 therebetween that follow the helical pattern of the cords 66. The pitch of the cords 66 is in a range of about 0.125 to about 0.225 (e.g., about 0.177). The braid 64 may compress the inner liner 60 over the mandrel 100. The braid 64 has an outer diameter in a range of about 0.052 to about 0.102 inches (e.g., 0.092 inches). It is contemplated that the inner liner 60, the braid 64, and the mandrel 100 may be supplied as a preassembled unit.

With reference to FIGS. 5 and 6, the first sensor 58 is formed by turning the wire 71 over the braid 64, or over the mandrel 100 and inner liner 60 just distal to the braid termination, transverse to a longitudinal axis of the braid to form turns 72 over the braid 64, or over the mandrel 100 and inner liner 60. Referring briefly back to FIG. 3, the turns 72 may include an inner layer 72a and an outer layer 72b of turns 72. Each layer of turns 72a, 72b may include a range of about 25 to about 200 individual turns 72 (e.g., about 100 individual turns) of the wire 71. The number of turns 72 in the inner layer 72a may be substantially equal to the number of turns 72 of the outer layer 72b.

As shown, the first sensor 58 includes two layers of turns 72a, 72b; however, it is contemplated that the first sensor 58 may include a single layer of turns 72 or may include more than two layers of turns 72. The number of turns 72 of the wire 71 is proportional to signal strength of the first sensor 58. That is, as the number of turns 72 increases, the signal strength of the first sensor 58 increases. As the number of layers of turns 72 is increased, the flexibility of the catheter 50 in the area of the turns 72 is reduced and the diameter of the catheter 50 in the area of the turns 72 is increased.

The total length of the layers 72a, 72b of the turns 72 is in a range of about 0.04 to about 0.36 inches (e.g., about 0.18 inches). As the total length of the layers 72a, 72b is increased, the flexibility of the catheter 50 in the area of the turns 72 is reduced. Thus, the number of layers of turns 72, the length of the turns 72, and the total number of turns 72 is a compromise between the signal strength of the first sensor 58 and the flexibility and size of the catheter 50.

The turns 72 are formed by ends or leads 76a, 76b of the wire 71 being turned around about the braid 64. The inner and outer layers 72a, 72b may be formed simultaneously by beginning at a distance spaced away from a distal end 65b of the braid 64. The inner layer 72a is formed by a first lead 76a being turned around the braid 64 in a first direction substantially transverse to a longitudinal axis of the braid 64 while proximally traversing an outer surface of the braid 64. The outer layer 72b is formed by a second lead 76b being turned around the braid 64 and the inner layer 72a in a second direction, opposite to the first direction, substantially transverse to a longitudinal axis of the braid 64 while proximally traversing the inner layer 72a. The turning of the second lead 76b trails the turning of the first lead 76a in a range of about 1 to about 5 turns 72 of the wire 71 (e.g., 2 turns) to allow the inner layer 72a to form before being covered by the outer layer 72b. The turns 72 begin spaced away from the distal end 65b of the braid 64 a distance in a range of about 0.0 to about 3.0 inches (e.g., about 0.5 inches). The turns 72 are spaced away a distance from the distal end 65b of the braid 64 large enough to ensure that the inner liner 60 and the braid 64 are substantially cylindrical in shape in the region of the turns 72 while minimizing the distance the turns 72 are space away from the distal end 65b to reduce wasted materials.

In embodiments where the first sensor 58 includes an odd number of turns 72, one lead exits the first sensor 58 at a proximal end of the first sensor 58 and the other lead exits the sensor at a distal end of the first sensor 58. The lead that exits the first sensor 58 at the distal end is placed under or over the turns 72 prior to wrapping the leads 76a, 76b over the braid 64 as detailed below.

Additionally, a support tube or layer may be positioned over the braid 64 before the wire 71 is wrapped over the braid 64 to form the first sensor 58 or may be placed over the first sensor 58 after the wire 71 is wrapped over the braid to form the first sensor 58. The support layer may be a ferro-metallic tube or a powder with resin that is configured to strengthen or support the first sensor 58 to prevent the first sensor 58 from deforming when used. The support layer may increase the signal strength of the first sensor 58 such that the length and/or number of turns 72 required to achieve a desired signal strength for the first sensor 58 may be reduced.

With particular reference to FIG. 6, the leads 76a, 76b are brought together within a channel 67 defined between two cords 66 of the braid 64 at a proximal end of the turns 72 to form a twisted pair 78. The leads 76a, 76b of the wire 71 are twisted together to form the twisted pair 78 to reduce or eliminate a signal from being generated by the wire 71 along the length of the braid 64 (i.e., utilizing the constructive interference to minimize the signal generated). The leads 76a, 76b are twisted together in a range of about 5 to about 15 twists per inch (e.g., about 10 twists per inch) of the wire 71. The twisted pair 78 of leads 76a, 76b is wrapped around the braid 64 within a channel 67 of the braid 64 such that the twisted pair 78 of leads 76a, 76b follows the pitch of the braid 64. Wrapping the twisted pair 78 of leads 76a, 76b helically around the braid 64 may increase the fatigue life of the leads 76a, 76b in response to flexing of the catheter 50 along its longitudinal axis. The twisted pair 78 of leads 76a, 76b is wrapped about the braid 64 from the turns 72 along a substantial length of the braid 64 towards a proximal end 65a of the braid 64. As shown, the twisted pair 78 of leads 76a, 76b are wrapped about the braid 64 in a clockwise direction when viewed from the proximal end 65a of the braid 64; however, the twisted pair 78 of leads 76a, 76b may be wrapped about the braid 64 in a counter-clockwise direction when viewed from the proximal end 65a of the braid 64. Forming the turns 72 and the twisted pair 78 of leads 76a, 76b with a single continuous wire increases the service life of the catheter 50 by eliminating the need for a connection (e.g., a soldered connection) between the turns 72 and each of the twisted pair 78 of leads 76a, 76b; however, in some embodiments, the turns 72 may be soldered to the twisted pair 78 of leads 76a, 76b

It is contemplated that a single device or a combination of devices may be used to turn the wire 71 about the braid 64 to form the turns 72, twist the pair of leads 76a, 76b of the wire 71 together, and wrap the twisted pair 78 of leads 76a, 76b of the wire 71 about the braid 64. Such a device for turning/wrapping the wire 71 is disclosed in U.S. patent application Ser. No. 15/879,087, filed Jan. 24, 2018, and entitled “METHODS OF MANUFACTURING A CATHETER HAVING A SENSOR.” Alternatively, it is contemplated that the turns 72 and the twisted pair 78 of leads 76a, 76b may be performed apart from the braid and then positioned or loaded over the braid 64. Once the turns 72 are loaded over the braid 64, the twisted pair 78 of leads 76a, 76b is wrapped about the braid 64 as detailed above.

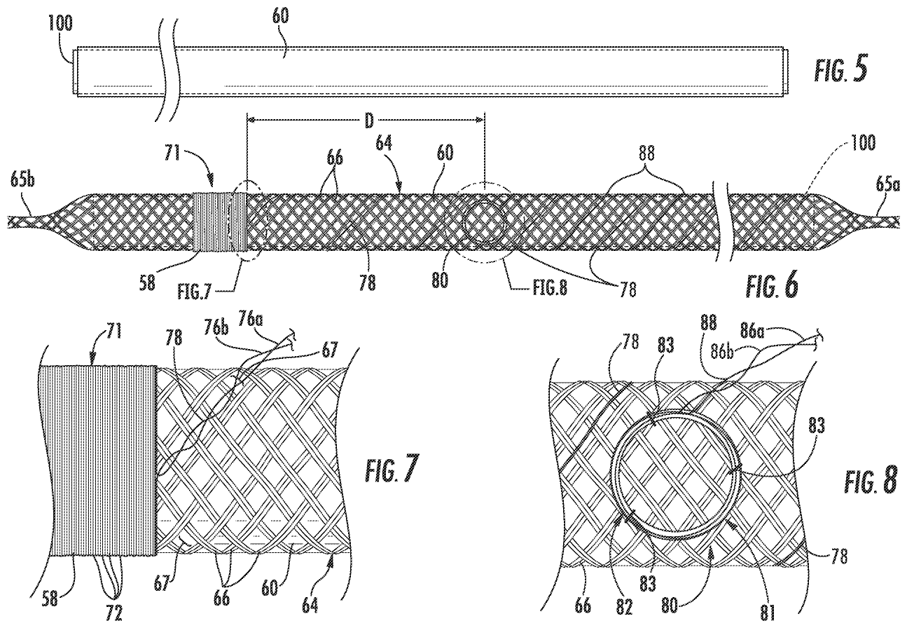

With particular reference to FIG. 8, the second sensor 80 is formed of a coil 82 of the wire 81 and is spaced proximally from the first sensor 58 a distance D (FIG. 6). The coil 82 is formed by turns of the wire 81 which form a loop and may include between 5 and 20 turns of the wire 81, e.g., 10 turns. The coil 82 is laid over the braid 64 in a position such that the coil 82 will be disposed between wraps of the twisted pair 78 of leads 76a, 76b.

The wire 81 includes two leads 86a, 86b that form a twisted pair 88 within a channel 67 defined between two cords 66 of the braid 64. The leads 86a, 86b of the wire 81 are twisted together to form the twisted pair 88 to reduce or eliminate a signal from being generated by the wire 81 along the length of the braid 64 (i.e., utilizing the constructive interference to minimize the signal generated). Forming the coil 82 and the twisted pair 88 of leads 86a, 86b with a single continuous wire increases the service life of the catheter 50 by eliminating the need for a connection (e.g., a soldered connection) between the coil 82 and each of the twisted pair 88 of leads 86a, 86b.

The leads 86a, 86b are twisted together in a range of about 5 to about 15 twists per inch (e.g., about 10 twists per inch) of the wire 81. The twisted pair 88 of leads 86a, 86b is wrapped around the braid 64 within a channel 67 of the braid 64 such that the twisted pair 88 of leads 86a, 86b follows the pitch of the braid 64. Wrapping the twisted pair 88 of leads 86a, 86b helically around the braid 64 may increase the fatigue life of the leads 86a, 86b in response to flexing of the catheter 50 along its longitudinal axis. The twisted pair 88 of leads 86a, 86b is wrapped about the braid 64 from the wraps 82 along a substantial length of the braid 64 towards a proximal end 65a of the braid 64. The twisted pair 88 of leads 86a, 86b are wrapped about the braid 64 in the same direction as the twisted pair 78 of leads 76a, 76b such that the twisted pair 88 is disposed between or interleaved with the twisted pair 78 along the braid 64 such that a diameter of the braid 64 is unchanged in areas having the twisted pairs 78, 88.

The coil 82 may be positioned on the braid 64 and the twisted pair 88 wrapped about the braid 64 simultaneously with the wire 71 forming turns 72 and the twisted pair 78 being wrapped about the braid 64 or the coil 82 may be positioned on the braid 64 and the twisted pair 88 wrapped about the braid 64 before or after the wire 71 forming turns 72 and the twisted pair 78 being wrapped about the braid 64.

In embodiments, the coil 82 is temporarily held in place relative to one or more of the cords 66 of the braid 64 by one or more ties 83. The ties 83 may be constructed of a variety of materials including conductive or non-conductive materials. The ties 83 may be constructed of a polymer that is melted into tubes or sleeves of the catheter, e.g., inner tube 69 or outer tube 70 detailed below. In some embodiments, the coil 82 is temporarily held in place relative to one or more of the cords 66 by adhesive that is placed on the coil 82 or the cords 66. In particular embodiments, the coil 82 is pressed into the braid 64 such that the coil 82 is held in place without adhesive or ties 83. In certain embodiments, the inner liner 60 flows through the braid 64 such that when the coil 82 is pressed into the braid 64, the coil 82 is temporarily held in place along the braid 64 by the inner liner 60.

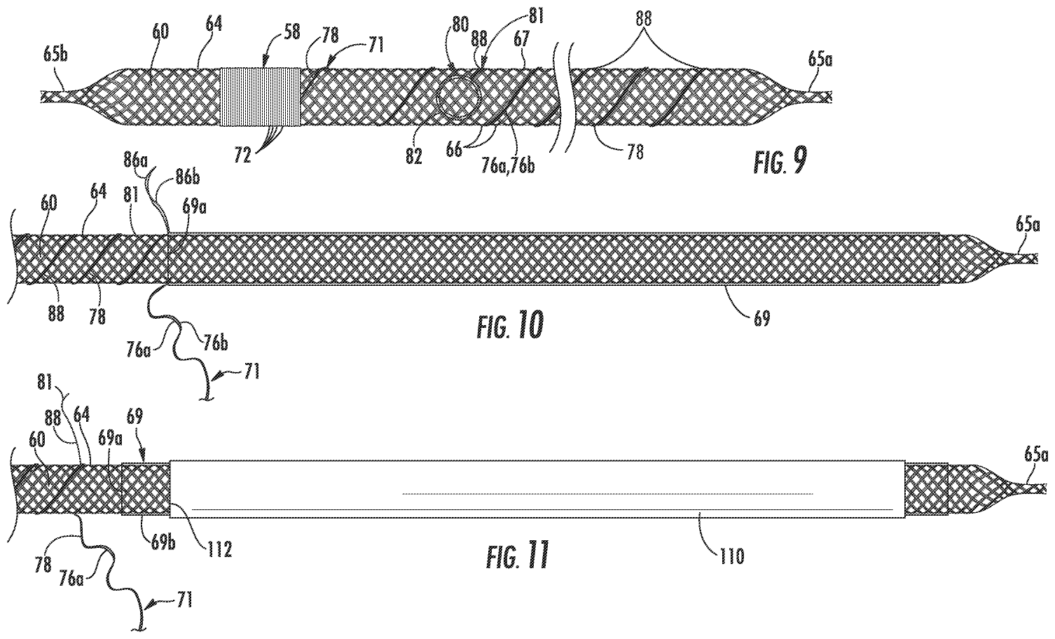

Referring now to FIG. 9, with the twisted pair 78 of leads 76a, 76b wrapped about the braid 64, the coil 82 temporarily held in place along the braid 64, and the twisted pair 88 is wrapped about the braid 64; a first outer tube 69 is slid over the proximal end 65a of the braid 64 until a proximal end 69a of the first outer tube 69 is adjacent to the twisted pairs 78, 88. The first outer tube 69 is a polymer tube which is then covered by heat shrink to melt or reflow the polymer such that the first outer tube 69 reflows or bonds to the braid 64. In addition, when the first outer tube 69 is reflowed, the inner liner 60 within the first outer tube 69 may be reflowed to bond with the braid 64 and the first outer tube 69. The outer diameter of the first outer tube 69 may be slightly reduced as the first outer tube 69 is reflowed.

With reference to FIG. 11, an outer sleeve 110 is slid over the reflowed first outer tube 69 until a distal end 112 of the outer sleeve 110 is adjacent a distal end 69a of the first outer tube 69. A portion 69b of the first outer tube 69 is exposed between the twisted pairs 78, 88 and the outer sleeve 110. The twisted pairs 78, 88 are then wrapped over the exposed portion 69b of the reflowed first outer tube 69 and over the outer sleeve 110 as shown in FIG. 12 with the twisted pairs 78, 88 interleaved with one another. The outer sleeve 110 is constructed of or lined with PTFE such that the outer sleeve 110 and the twisted pairs 78, 88 are prevented from bonding to a portion of the reflowed first outer tube 69 covered by the outer sleeve 110.

Referring to FIG. 13, a second outer tube 70 is slid over the distal end 65b of the braid 64, the first sensor 58, the coil 82 of the second sensor, the twisted pairs 78, 88, the exposed portion 69b of the first outer tube 69, and a portion of the outer sleeve 110. Similar to the first outer tube 69, the second outer tube 70 has an inner diameter slightly larger than the braid 64 such that the second outer tube 70 freely slides over the braid 64, the wire 71, the wire 81, the reflowed first outer tube 64, and the outer sleeve 110. A proximal end 70a of the second outer tube 70 is positioned along the outer sleeve 110. The second outer tube 70 is a polymer tube which is then covered by heat shrink to reflow the polymer such that the second outer tube 70 bonds to the braid 64 and to the exposed portion 69b of the first outer tube 69. In addition, when the second outer tube 70 is reflowed, the inner liner 60 and the first inner tube 69 within the second outer tube 70 may be reflowed to bond with the braid 64, the second outer tube 70, the wire 71, and the wire 81. After the second outer tube 70 is reflowed, the first and second outer tubes 69, 70 form the outer coating 68 as a continuous layer of polymer over the braid 64 and a substantial portion of the wires 71, 81. The outer coating 68 is substantially smooth and seals the EWC 64 (FIG. 3). Further, as the second outer tube 70 is reflowed, the coil 82 is secured in place about the braid 64. In embodiments, as the second outer tube 70 is reflowed, elements which temporarily held the coil 82 in place, e.g., adhesive or ties 83, may flow into the second outer tube 70 or be eliminated from the construct. As shown, the outer coating 68 is translucent or transparent; however, the outer coating 68 may be opaque and have a desired color (e.g., blue or white).

With particular reference to FIG. 14, with the outer coating 68 formed, the twisted pairs 78, 88 are unwrapped from over the outer sleeve 110 in a manner not to damage the leads 76a, 76b, 86a, 86b. Then the outer sleeve 110 is peeled off or removed from over the portion of the outer coating 68 previously formed from first outer tube 69. Any portion of the second outer tube 70 that is formed over the outer sleeve 110 is separated from the outer coating 68 and discarded.

Referring to FIG. 15, the catheter 50 is cut adjacent the proximal end 65a of the braid 64 to define a proximal end 53 of the catheter 50. The catheter 50 is cut a distance spaced away from a point 77 where the leads 76a, 76b, 86a, 86b pass through the outer coating 68 to leave a proximal portion 52 of the catheter 50 where the leads 76a, 76b, 86a, 86b are not wrapped around the braid 64. The catheter 50 is also cut adjacent the first sensor 58 to define the distal end 55 of the catheter 50. The cut adjacent the first sensor 58 is spaced apart a minimal distance from the first sensor 58 to prevent discontinuities in the wire 71 forming the first sensor 58; however, this distance is minimized to allow the first sensor 58 to be positioned as close to the distal end 55 of the catheter as possible. The distal end 55 of the catheter 50 may be reflowed to seal the portions of the braid 64 exposed by cutting adjacent the sensor. Additionally or alternatively, a catheter tip (not shown) may also be bonded to the distal end 55 of the catheter 50. After the catheter 50 is cut, the catheter 50 has a total length of about 30 to about 90 inches (e.g., about 60 inches) from the proximal end 53 to the distal end 55.

Still referring to FIG. 15, after the catheter 50 is cut to length, a hub 90 is positioned over the proximal portion 52 of the catheter 50. The hub 90 defines a channel 91 therethrough and includes a proximal guide portion 92, a central collar 94, and a distal collar 96. The proximal portion 52 of the catheter 50 is positioned within the channel 91 of the hub 90 such that the proximal end 53 of the catheter is adjacent the central collar 94 of the hub 90. The hub 90 is then bonded to the proximal portion 52 of the catheter 50. The hub 90 may be reflowed into the outer coating 68 of the catheter 50 or bonded to the outer coating 68 using adhesives. The proximal guide portion 92 is configured to guide an instrument (not shown) into the EWC (FIG. 3) of the catheter 50.

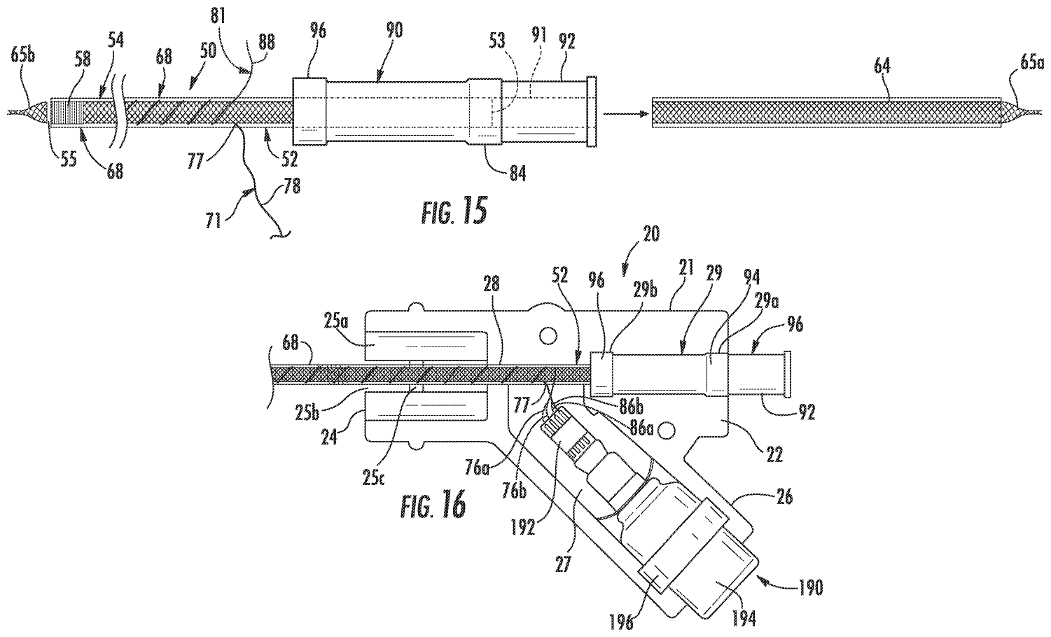

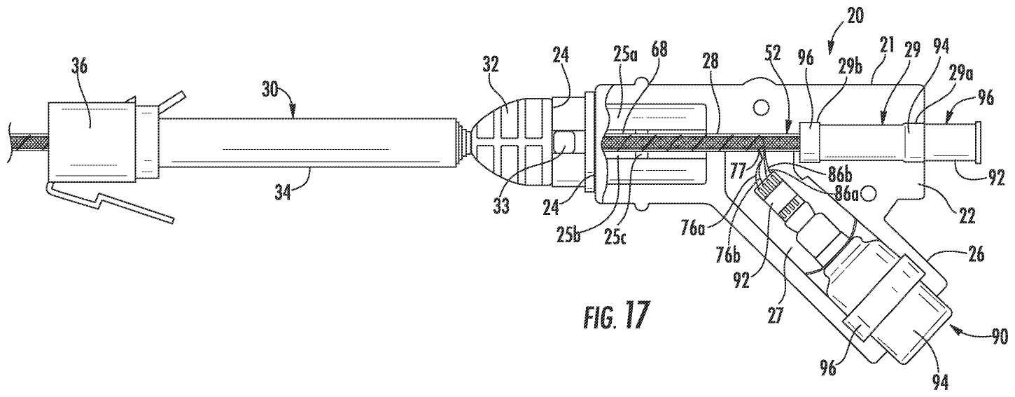

With particular reference to FIG. 16, the handle assembly 20 is coupled over the proximal portion 52 of the catheter 50. The handle assembly 20 includes a body 21 formed from two half shells coupled together over the proximal portion 52 of the catheter 50. Each of the half shells of the body 21 is substantially similar to one another and is joined along the longitudinal axis of the catheter 50. The body 21 has a proximal end 22, a distal end 24, and a connector portion 26. The body 21 defines a catheter passage 28 that is configured to receive the catheter 50 with the hub 90 bonded to the distal end 52. The catheter passage 28 includes an enlarged proximal portion 29 including a first recess 29a and a second recess 29b. The proximal portion 29 is sized to receive the hub 90 to longitudinally fix the catheter 50 relative to the handle 20. The first recess 29a is sized to receive the central collar 94 of the hub 90 and the second recess 29b is sized to receive the distal collar 96 of the hub 90. The proximal portion 29 of the catheter passage 28 between the first and second recesses 29a, 29b has a diameter smaller than the first and second recesses 29a, 29b to fix the hub 90 within the catheter passage 28. The hub 90 is frictionally engaged with the body 21 to rotatably secure the hub 90 within the catheter passage 28. It is contemplated that the hub 90 may be adhered to the body 21 within the catheter passage 28.

With continued reference to FIG. 16, the handle 20 includes an electrical coupling 190 disposed within a cavity 27 of the connector portion 26 of the handle 20. The electrical coupling 190 includes an internal connector 192, an external connector 194, and a collar 196 positioned between the internal and external connectors 192, 194. The collar 196 is received by the connector portion 26 of the handle 20 to form a seal to isolate the internal connector 192 within the cavity 27 of the connector portion 26. The cavity 27 of the connector portion 26 is isolated from the external environment by a seal formed by the walls defining the catheter passage 28 engaging the outer coating 68 of the catheter 50 and the seal formed by the collar 196 engaging walls defining the cavity 27 of the connector portion 26.

The point 77 at which the twisted pairs 78, 88 exit through the outer coating 68 of the catheter 50 is positioned within the cavity 27 of the connector portion 26. The leads 76a, 76b and leads 86a, 86b are untwisted from one another and electrically coupled (e.g., soldered) separately to the internal connector 192. The internal connector 192 is in electrical communication with the external connector 194 such that an adapter (not shown) may be coupled to the external connector 194 to supply electrical energy to the first sensor 58 and the second sensor 80 (FIG. 1) through the external connector 194 via the wires 71, 81 respectively. Examples of systems and methods for using the first and second sensors 58, 80 to guide the catheter 50 to targeted tissue may be found in commonly assigned U.S. Pat. Nos. 9,459,770 and 9,247,992.

With particular reference to FIG. 17, the distal end 24 of the body 21 defines an opening 25a configured to receive the first end 32 of the telescopic channel 30. The body 21 may define a slot 25b parallel to the catheter passage 28 and in communication with the opening 25a to receive a clip 33 of the first end 32 of the telescopic channel 30 to rotatably fix the first end 32 of the telescopic channel 30 to the body 21. The distal end 24 of the body 21 may also include a ring 25c positioned about an outer surface of the body 21 transverse to the longitudinal axis of the catheter passage 28 and over the slot 25b. The clip 33 of the first end 32 of the telescopic channel 30 may engage the ring 25c when the clip 33 is disposed within the slot 25b to longitudinally the first end 32 of the telescopic channel 30 to the body 21 of the handle 20. The telescopic channel 30 may be attached a bronchoscope (not shown) and the catheter 50 of the catheter assembly 10 may be inserted through the first end 32 of the telescopic channel 30 until the distal end 24 of the handle 20 receives the first end 32 of the telescopic channel 30.

As detailed above, the catheter 50 is constructed over a braid 64. However, in some embodiments, the catheter 50 does not include the braid 64 and is formed of the liners and tubes that are flowed into one another to form a catheter 50 of unitary construction having first and second sensors 58, 80. Further, as detailed above, the handle assembly 20 is coupled to a proximal portion of the catheter 50; however, the catheter 50 may have a variety of handle assemblies 20 or no handle assembly 20 coupled to the proximal portion of the catheter 50.

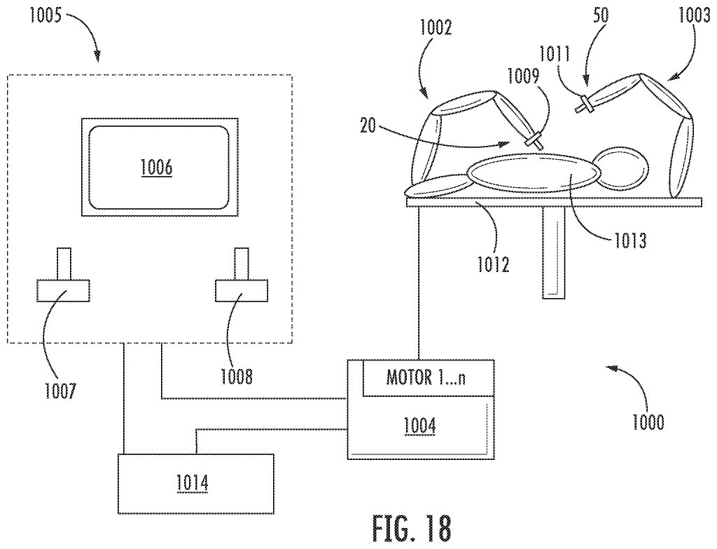

Referring to FIG. 18, a medical work station is shown generally as robotic system or work station 1000 and generally may include a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a clinician, may be able to telemanipulate robot arms 1002, 1003 in a first operating mode.

Each of the robot arms 1002, 1003 may include a plurality of members, which are connected through joints, and an attaching device 1009, 1011, to which may be attached, for example, the catheter 50 and/or the handle assembly 20, in accordance with any one of several embodiments disclosed herein.

Robot arms 1002, 1003 may be driven by electric drives (not shown) that are connected to control device 1004. Control device 1004 (e.g., a computer) may be set up to activate the drives, in particular by means of a computer program, in such a way that robot arms 1002, 1003, the attaching devices 1009, 1011, e.g., the catheter 50 and/or the handle assembly 20, execute a desired movement according to a movement defined by means of manual input devices 1007, 1008. Control device 1004 may also be set up in such a way that it regulates the movement of robot arms 1002, 1003 and/or of the drives.

Medical work station 1000 may be configured for use on a patient 1013 lying on a patient table 1012 to be treated in a minimally invasive manner by means of the localization system. Medical work station 1000 may also include more than two robot arms 1002, 1003, the additional robot arms likewise being connected to control device 1004 and being telemanipulatable by means of operating console 1005. Medical work station 1000 may include a database 1014, in particular coupled to with control device 1004, in which are stored, for example, pre-operative data from patient/living being 1013 and/or anatomical atlases.

For a more detailed description of the construction and operation of an exemplary robotic surgical system reference is made herein to U.S. Pat. No. 8,828,023.

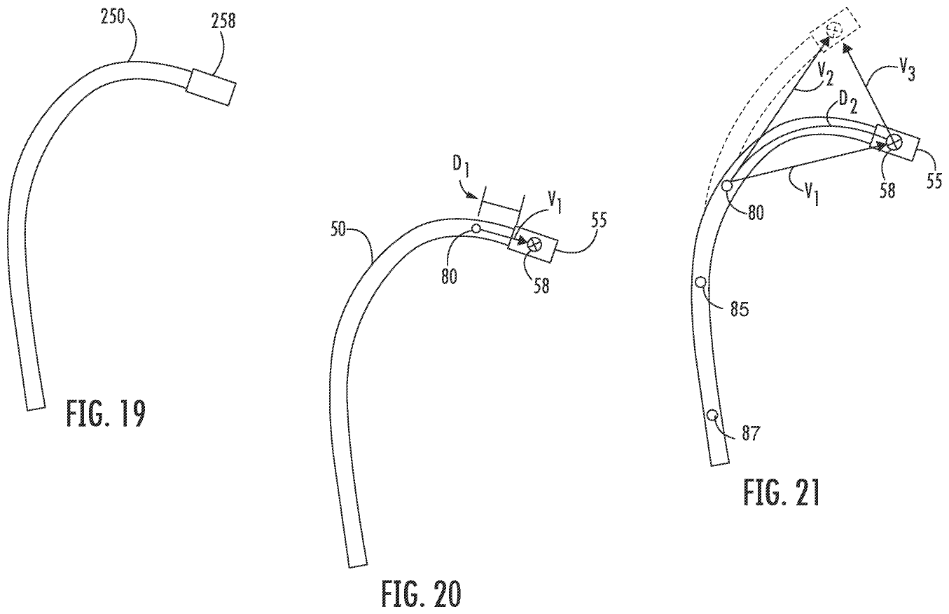

Referring now to FIG. 19, the prior art catheter 250 is shown with a single sensor 258. When the single sensor 258 is used to detect the position of the prior art catheter 250, the single sensor 258 can only account for 5 Degrees-of-Freedom (DOF) of the prior art catheter 250. Specifically, the single sensor 258 does not provide an indication of the rotation of the prior art catheter 250.

With reference to FIG. 20, the catheter 50, detailed above, is provided with the second sensor 80 disposed a distance D₁ along the catheter 50 from the first sensor 58. The distance D₁ is relatively small such that the second sensor 80 is spaced apart from the first sensor 58 a short distance in a range of about 0.25 inches to about 1 inch. As the distance D₁ is known and is small, the catheter 50 between the first and second sensors 58, 80 is substantially rigid such that a vector V₁ from the second sensor 80 to the first sensor 58 determines an orientation of catheter 50 such that rotation of the catheter 50 can be determined. By determining the rotation of the catheter 50, the catheter 50 can be tracked through 6 DOF.

Referring now to FIG. 21, the catheter 50 detailed above, is provided with the second sensor 80 disposed a distance D₂ along the catheter 50 from the first sensor 58. The distance D₂ is larger than the distance D₁ such that the catheter 50 is flexible between the first and second sensors 58, 80. The distance D₂ may be in a range of about 2 inches to about 4 inches. When the first sensor 58 is spaced apart from the second sensor 80 the distance D₂, the change in a vector from the second sensor 80 to the first sensor 58, a deflection of the catheter 50 between the first and second sensors 58, 80 is detectable. In addition, the entire arc or curve of the catheter can be determined between the second sensor 80 and the first sensor 58. For example, when the distal end 55 of the catheter 50 is in a first position P₁, the vector V₁ defines the position of the first sensor 58 relative to the second sensor 80 and when the distal end 55 of the catheter 50 is in the second position P₂, the vector V₂ defines the position of the first sensor 58 relative to the second senor 80 such that a vector V₃ defines the deflection of the distal end 55 of the catheter 50 such that the torsional behavior of the catheter 50 can be determined such that the catheter 50 can be tracked through 6 DOF.

In aspects, the catheter 50 may include more than two sensors, such as third and fourth sensors 85, 87 spaced along the length of the catheter 50. The catheter 50 may include any suitable number of sensors, such as four or more. The third and fourth sensors 85, 87 may be disposed proximally of the first and second sensors 58, 80. The third and fourth sensors 85, 87 enable a determination of the entire catheter shape from the catheter tip 55 to the proximal-most sensor. The 5 DOF of each sensor 58, 80, 85, 87 may be determined along with the distance between each of the sensors 58, 80, 85, 87.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

1. (canceled)

2. A catheter comprising:
an elongated body having a proximal portion and a distal portion, the elongated body including a working channel defined therein;
a first sensor formed of a first wire, the first sensor disposed on the distal portion of the elongated body and configured to detect a position of a distal end of the elongated body within a patient; and
a second sensor disposed on the distal portion of the elongated body proximal to the first sensor, wherein a vector from a detected position of the second sensor to a detected position of the first sensor defines a rotational orientation of the elongated body.

3. The catheter according to claim 2, wherein the elongated body is substantially rigid between the first and second sensors.

4. The catheter according to claim 2, wherein the first and second sensors are separated by between 0.25 and 1.00 inch.

5. The catheter according to claim 2, wherein the first sensor is formed from turns of the first wire around elongated body, the first wire including first and second leads that are formed in a first twisted pair proximal of the turns of the first wire, the first twisted pair extending to the proximal portion of the elongated body.

6. The catheter according to claim 5, wherein the second sensor is formed from a coil of a second wire, the second wire including third and fourth leads that are formed in a second twisted pair proximal of the coil of the second wire, the second twisted pair extending to the proximal portion of the elongated body.

7. The catheter according to claim 6, wherein the first twisted pair is helically wound about the working channel between the first sensor and the proximal portion of the elongated body.

8. The catheter according to claim 7, wherein the coil is disposed within a wall of the elongated body and positioned between two adjacent helical wraps of the first twisted pair.

9. The catheter according to claim 8, wherein the second twisted pair is helically wound about the working channel between the second sensor and the proximal portion of the elongated body, the second twisted pair interleaved with the first twisted pair along the elongated body, wherein first and second wires are disposed entirely within the wall of the elongated body distal of the proximal portion.

10. The catheter according to claim 8, wherein the second twisted pair is helically wound about the working channel between the second sensor and the proximal portion of the elongated body, the second twisted pair interleaved with the first twisted pair along the elongated body, wherein the elongated body includes a braid that defines the working channel and an outer coating disposed over the braid for isolating the working channel from an environment surrounding the catheter.

11. The catheter according to claim 2, further comprising a third sensor formed of a third wire and disposed on the elongated body proximally of the second sensor.

12. the catheter according to claim 11, wherein the third sensor is configured to determine an arc of the catheter between the third sensor and the second sensor or between the third sensor and the first sensor.

13. A catheter system comprising:
a catheter including:
an elongated body having a proximal portion and a distal portion, the elongated body including a working channel defined therein;
a first sensor formed of a first wire disposed on the distal portion of the elongated body and configured to detect a position of a distal end of the elongated body within a patient;
a second sensor disposed on the distal portion of the elongated body proximal to the first sensor, wherein a vector from a detected position of the second sensor to a detected position of the first sensor defines a rotational orientation of the elongated body; and
a handle longitudinally and rotatably fixed to the catheter, the handle including an adapter in electrical communication with the first and second sensors.

14. The catheter system according to claim 13, further comprising a telescopic channel disposed over a portion of the elongated body and coupled to a distal end of the handle.

15. The catheter system according to claim 13, wherein the elongated body is substantially rigid between the first and second sensors.

16. The catheter system according to claim 13, wherein the first and second sensors are separated by between 0.25 and 1.00 inch.

17. The catheter system of claim 13, wherein the first sensor is formed from turns of the first wire around elongated body, the first wire including first and second leads that are formed in a first twisted pair proximal of the turns of the first wire, the first twisted pair extending to the proximal portion of the elongated body.

18. The catheter system of claim 17, wherein the second sensor is formed from a coil of the second wire, the second wire including third and fourth leads that are formed in a second twisted pair proximal of the coil of the second wire, the second twisted pair extending to the proximal portion of the elongated body.

19. The catheter system of claim 18, wherein the coil is disposed within a wall of the elongated body and positioned between two adjacent helical wraps of the first twisted pair.

20. The catheter system of claim 13, further comprising a third sensor formed of a third wire disposed on the elongated body a known distance proximally of the second sensor.

21. The catheter system of claim 20, wherein the third sensor is configured to determine an arc of the elongated body between the third sensor and the second sensor or between the third sensor and the first sensor.
